(19) United States
(12) Patent Application Publication
(10) Pub. No.: US 2003/0064504 A1
(43) Pub. Date: Apr. 3, 2003

(54) Intermediate guide element

(76) Inventors: Thomas Rothmann, Langenfeld (DE); Andreas Schafer, Leverkusen (DE); Ulf-Ingo Friederichs, Hombrechtikon (CH); Thi My Chi Nguyen, Dusseldorf (DE)

Correspondence Address: LEON R. YANKWICH, YANKWICH & ASSOCIATES, 201 BROADWAY, CAMBRIDGE, MA, 02139, US

(21) Appl. No.: 10/217,221

(22) Filed: Aug. 12, 2002

(30) Foreign Application Priority Data
Aug. 13, 2001 (DE) 10139758.5

(51) Int. Cl. C12M 1/34; B32B 27/04; B32B 27/12

(52) U.S. Cl. 435/287.1; 422/100

(57) ABSTRACT

Intermediate guide elements for use in a preparation apparatus for preventing cross-contamination and reducing the formation of residues in the feed lines are provided herein. The intermediate guide element has a main block with a desirable number of through-openings traversing the main block, preferably arranged parallel to one another. Preferably, the guide element has an inlet region and an outlet region, the inlet region being attachable to an end region of a feed line and the outlet region have a guide for each individual through-openings.

### TECHNICAL FIELD

The invention relates to an intermediate guide element for use in a preparation apparatus for preferably fully automated preparation of liquids, especially lysates, and a preparation apparatus. The intermediate guide element serves to channel and convey the liquid from one or more feed lines through a hotplate, for example, and thus on the one hand reduces the risk of cross-contamination particularly when a plurality of pipes are arranged side by side, and on the other hand also largely prevents the formation of residues in the feed line system.

### BACKGROUND OF THE INVENTION

Molecular biological methods of recovery and preparation, such as for example the washing and extraction of nucleic acid or the isolation of RNA and/or DNA, are often fully automated nowadays. Preferably, the starting liquid is generally placed in finely metered amounts in a plurality of containers, especially test tubes, arranged side by side, where it is washed and filtered. The filtering process is usually carried out using suitable filter elements which are arranged in the containers, mostly in the region of the bases of the containers. A vacuum can be created in the containers underneath the filters by means of an opening in the underside of the containers, by means of which the solution above the filters is sucked through the filters and thus filtered. In the recovery of DNA, for example, the DNA is adsorbed by means of a suitable membrane. Unfiltered residues can be eliminated in a rinsing operation. After subsequent elution of the DNA, the desired pure DNA is obtained.

For carrying out analytical and recovery processes of this kind, the “BioRobot 8000” system produced by the present Applicant has been on the market for some years. The system 10 shown in FIG. 1 essentially comprises a distribution device for supplying and distributing liquid (not shown in FIG. 1), a multi-tube rack 20, a hotplate 30 provided with a plurality of through-bores 32 and a suction device 40 for producing a vacuum. The multi-tube rack 20, which is also referred to as the rack, for simplicity's sake, comprises a plurality of test tubes 22 (modules) arranged side by side in which suitable filter elements 24 are integrated close to the bottom. In addition, the test tubes 22 each have openings 26 on their underside through which the pressure is equalised between the area around the test tubes and the interior of the test tubes underneath the filter elements. If a vacuum is then produced underneath the hotplate by means of the suction device, underpressure will also be formed in the region above the hotplate 30 as a result of the through-bores 32 provided in the hotplate 30, and in the interior of the test tubes underneath the filter elements as a result of the openings 24 provided in the test tubes 22, resulting in the liquids fed into the test tubes, the so-called lysate, being sucked through the filter elements. In the system shown, the filtered liquid passes through the openings provided in the test tubes 22 and out through the bottom of the test tubes and drips down towards the hotplate 30. Some of the liquid drips directly through the openings 32 in the hotplate 30 into one or more collecting containers provided underneath. However, another fraction of the liquid drips onto the hotplate 30 and remains there. If the multi-tube rack 20 is then lowered onto the hotplate 30 for drying and comes into contact with the hotplate 30 there is a danger that the bottoms of the test tubes 22 will be contaminated with the lysate residues on the hotplate. As the lysate on the hotplate may also run to some extent there will thus be cross-contamination of lysate residues from one test tube to another.

Moreover, it has also been found that, as shown in FIG. 2, in the existing system, after the lysate 60 or the washing buffer has been sucked through the filters 52 of the multi-tube rack (steps a) to b) in FIG. 2), lysate residues 62 or residues of washing buffer are left behind in the rims of the individual test tubes 50 and do not totally drain away from the test tubes. After the drying process (steps b) to c) in FIG. 2), salt residues 64 are therefore visible on the rim of the test tubes 50, which may also lead to contamination of the eluate subsequently produced.

### SUMMARY OF THE INVENTION

The problem of the invention is therefore on the one hand to provide an element which, in conjunction with the preparation apparatus described above, largely avoids the abovementioned problems. In particular, using the element according to the invention, it is intended to reduce the risk of cross-contamination which previously existed in automated sample preparation, particularly when using multi-tube racks, and expediently also to reduce the formation of residues inside the test tubes. The invention also sets out to provide a preparation apparatus which avoids the problems of the prior art.

This problem is solved according to the invention by an intermediate guide element for use in a preparation apparatus. The intermediate guide element comprises for this purpose a main block with a top and a bottom and at least one through-openings. Preferably, the intermediate guide element has a plurality of through-openings, preferably arranged parallel to one another, so as to allow the simultaneous preparation of a plurality of samples side by side in one procedure. Each of the through-openings extends continuously from the top of the main block to the bottom. Preferably, the through-openings of the intermediate guide element are simply constructed as bores. Moreover, each through-opening has an inlet region and an outlet region, the inlet region being constructed so that the inlet region can be attached to an end region of a feed line. Appropriately, to do this, the end region of the feed line can be inserted in the associated through-opening in an overlapping arrangement. Attached to the outlet region of each through-openings is a singular guide element which is constructed as a tube in a preferred embodiment and which preferably fits into a section of the outlet region of the associated through-opening. The singular guide element projects beyond the main block of the intermediate guide element by a length which corresponds to at least one thickness of the hotplate arranged in the preparation apparatus on the bottom of the main block of the intermediate guide element. In the “BioRobot 8000” system described above the intermediate guide element may be disposed between the multi-tube rack and the hotplate. Each outlet of a test tube in the multi-tube rack then opens directly into a through-opening, so that any lysate leaving the tube goes directly into a through-opening. In addition, the intermediate guide element is arranged on the hotplate so that the singular guide elements each pass through the hotplate, preferably through one of the through-bores provided in the hotplate. Lysate leaving the through-openings in the intermediate guide element thus passes over the associated singular guide element directly into the region below the hotplate, thereby ruling out any contamination of the hotplate with lysate residues.

In one appropriate embodiment the inlet region is stepped, preferably so that it precisely fits the contour of the end region of the associated feed line.

On the one hand self-adjustment is obtained during the fitting on and insertion of the end regions of the feed lines, which are in the form of tubes in the “BioRobot 8000” system, as a result of the guiding action of the stepped contour of the inlet region. On the other hand, it has also been found that an inlet region of this configuration both provides an optimum seal for the lysate leaving the feed lines and also, as a result of the capillary effect, reduces or even totally prevents the formation of residues in the region of the feed lines.

Moreover, the main block of the intermediate guide element expediently has an encircling rim on top and preferably at least one centring recess, more particularly two centring recesses. Both the rim and the optional centring recesses serve to adjust the intermediate guide element when it is used in a preparation apparatus and thus allow easier manipulation.

The preparation apparatus according to the invention particularly for the fully automated preparation of liquid samples such as lysate samples comprises a feed element with one or more feed lines, as well as an intermediate guide element as described above and a hotplate. The intermediate guide element releasably mounted in the preparation apparatus has a number of through-openings corresponding to the number of feed lines in the feed region and is adjacent to the feed element on the top of the main block. The hotplate is disposed on the bottom of the main block and comprises a number of through-bores corresponding to the number of singular guide elements. For this purpose, the through-bores are preferably arranged in the hotplate in such a way that in the assembled preparation apparatus each through-bore is aligned with one singular guide element.

The intermediate guide element is preferably capable of being positioned on the hotplate of the preparation apparatus and removed therefrom fully automatically by means of a gripping and positioning device. Moreover, in one particular embodiment, the feed element may also be fully automatically adjustable in height and in a particularly advantageous embodiment it is also replaceable. Thus, the preparation apparatus can be assembled fully automatically or individual elements can be replaced fully automatically. In conjunction with other fully automatically controlled equipment known from the prior art, such as, for example, gripping means for changing the feed element or a fully automatic dispensing device for finely metered and time-controlled feeding of amounts of liquid such as lysate or washing solution, for example, to the individual feed lines, fully automated sample preparation is possible, even over a plurality of successive sampling cycles.

In a preferred embodiment of the preparation apparatus the feed element is constructed as a single or multiple tube rack. The provision of a plurality of test tubes side by side makes it possible to prepare a plurality of samples at the same time, while if a single tube rack is used the samples can only be prepared sequentially. Preferably, filters for filtering the liquid are disposed in the test tubes. Expediently, a vacuum can also be applied to the side of the hotplate remote from the intermediate guide element, so that the sample liquid can be sucked out of the feed region through the filters.

### BRIEF DESCRIPTION OF THE DRAWINGS

The invention is hereinafter explained more fully with reference to some embodiments by way of example, in conjunction with the drawings, wherein: FIG. 1 is a three-dimensional view of the basic structure of the “BioRobot 8000” system known in the art;

FIG. 2 is a diagrammatic view of the formation of residues in a test tube in the multi-tube rack in the “BioRobot 8000” system during the filtering and drying of the lysate;

FIG. 3 is a three-dimensional view of an intermediate guide element according to the invention;

FIG. 4 is a cross-sectional representation of a preparation apparatus according to the invention when using an intermediate guide element according to the invention;

FIG. 5 shows the results of samples obtained with a preparation apparatus known from the prior art;

FIG. 6 shows the results of samples obtained with a preparation apparatus according to the invention.

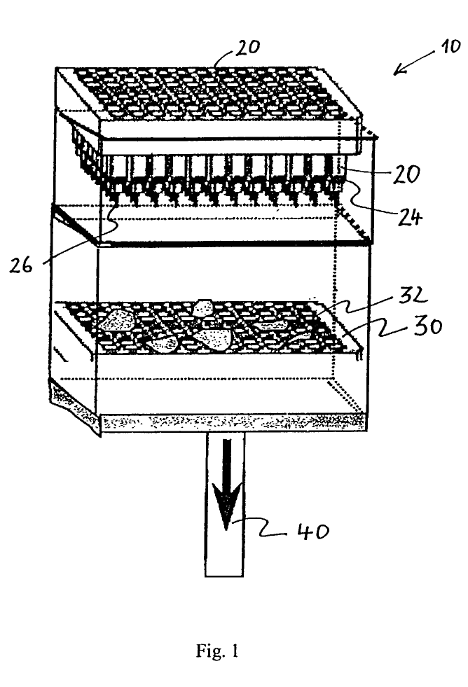

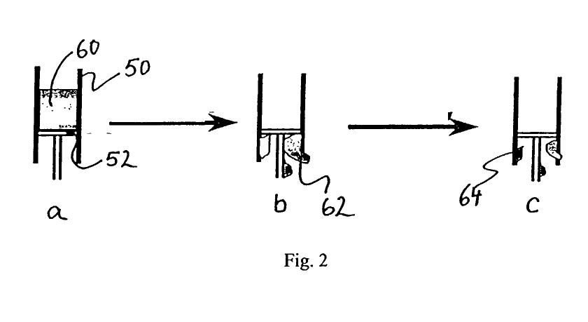

In the Figures, only the elements and components essential to an understanding of the invention are shown. The apparatus shown according to the invention may thus be expanded in a variety of ways or modified in a manner obvious to the skilled man without departing from or modifying the concept of the invention. Identical or equivalent components have mainly been marked with the same reference numerals in the Figures.

DETAILED DESCRIPTION

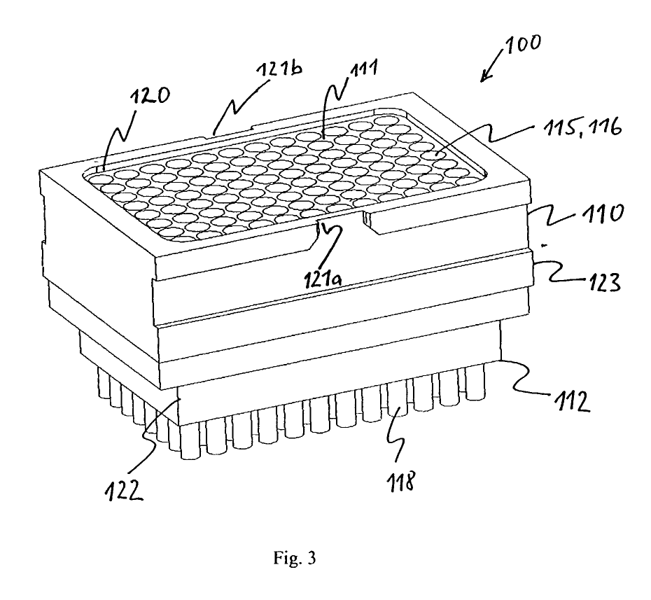

FIG. 3 shows a three-dimensional view of an intermediate guide element 100 according to the invention. The width of the intermediate guide element 100 is in the range from 100 mm to 150 mm, preferably in the range from 125 mm to 135 mm, more preferably 130 mm. The depth of the intermediate guide element is in the range from 70 mm to 110 mm, preferably in the range from 85 mm to 95 mm, most preferably 90 mm. The height of the intermediate guide element is in the range from 65 mm to 95 mm, preferably in the range from 70 mm to 80 mm, most preferably 75 mm. The intermediate guide element 100 comprises a main block 110 with a top 111 and a bottom 112. Moreover, 96 through-openings 115 arranged parallel to one another extend in the main block 110 of the intermediate guide element 100 shown here from the top 111 to the bottom 112 of the main block 110. The through openings 115 are simply constructed as through-bores.

In the inlet regions 116 of the through-openings 115 directed towards the top 111 of the main block 110 the through-openings 115 are each enlarged in diameter by means of a countersunk bore so that the bore has a stepped contour when viewed in cross section. This stepped contour of the inlet regions 116 of the through-openings 115 on the one hand facilitates the insertion and adjustment of feed lines. On the other hand, when an appropriate feed line is attached thereto, the end region of which is preferably constructed to have a mirror-symmetrical contour corresponding to the stepped contour, the feed line system formed has a fluidtight connection to the through-opening with a smooth inner contour.

In the embodiment of the intermediate guide element 100 shown, tubes 118 are inserted as the singular guide elements in the outlet regions of the through-openings 115 directed towards the underside 112 of the main block 110 (not visible in FIG. 3 because of the perspective view). The diameters of the outlet regions 117 of the through-openings 115 are also enlarged with a stepped configuration by means of a countersunk bore, each tube 118 fitting exactly into the outlet region 117 of a through-opening 115, so that here too the feed line system formed has a fluidtight and smooth inner contour. The tubes 118 protrude from the main block 110 of the intermediate guide element 100 by a length corresponding to 1.3 times the thickness of a hotplate.

Moreover, the intermediate guide element 100 is constructed on the top 111 with an encircling rim 120. This, and the two centring recesses 121a, 121b lying opposite each other along the longitudinal sides, serve to adjust and align the feed device disposed in a preparation apparatus on the top 111 of the intermediate guide element 100.

The step 122 provided on the underside 112 serves to adjust and align the intermediate guide element 100 in relation to a hotplate provided in a preparation apparatus on the bottom 112 of the intermediate guide element 100.

The central thickening 123 around the main block 110 of the intermediate guide element 100 also assists with the gripping and positioning of the intermediate guide element 100, expediently by means of a fully automatically controlled gripping and positioning device.

The intermediate guide element 100 shown in FIG. 3 may be used, for example, in a “BioRobot 8000” apparatus known in the art (designated 10 in FIG. 1) and reliably prevents the problems of cross-contamination between different samples known to occur in the prior art, particularly during the drying process, as a result of the feed element in the form of a multi-tube rack 20 being placed on the hotplate 30 and essentially also the formation of residues in the individual test tubes 22 of the multi-tube rack. For this purpose, the intermediate guide element 100 according to the invention, shown in FIG. 3, is positioned between the hotplate 30 and the multi-tube rack 20, the bottom 112 of the main block 110 of the intermediate guide element 100 being adjacent to the hotplate 30 while the top 111 is adjacent to the multi-tube rack 20. The through-bores 32 provided in the hotplate 30 are aligned so as to register with the tubes 118 mounted on the bottom 112 of the intermediate guide element 100, so that when the intermediate guide element 100 is arranged directly on the hotplate 30 the tubes 118 project beyond the underside of the hotplate by 0.3 times the thickness of the hotplate. Moreover, when the intermediate guide element 100 is used, each test tube in the multi-tube rack 20 opens into a separate through-opening 115 of the intermediate guide element 100.

Any liquid leaving the tubes 118 of the multi-tube rack 20 thus passes directly into a through-opening 115 in the intermediate guide element 100 and from there into a tube 118 arranged on the underside of the intermediate guide element 100. The draining of the liquid from the particular tube 118 takes place underneath the hotplate 30, so that the hotplate 30 is not contaminated with escaping liquid.

Moreover, the capillary action of the through-openings 115 in the intermediate guide element 100 leads to an at least partial reduction in the formation of residues in the outlet region of the test tubes 22 in the multi-tube rack 20.

There is no need to modify the operation of the “BioRobot 8000” system 10, as the intermediate guide element 100 is removable. After manual or automatic removal of the intermediate guide element 100 the multi-tube rack 20 may be lowered onto the hotplate 30 for drying. Because the surface of the hotplate 30 is now clean, there can be no cross-contamination between the individual samples.

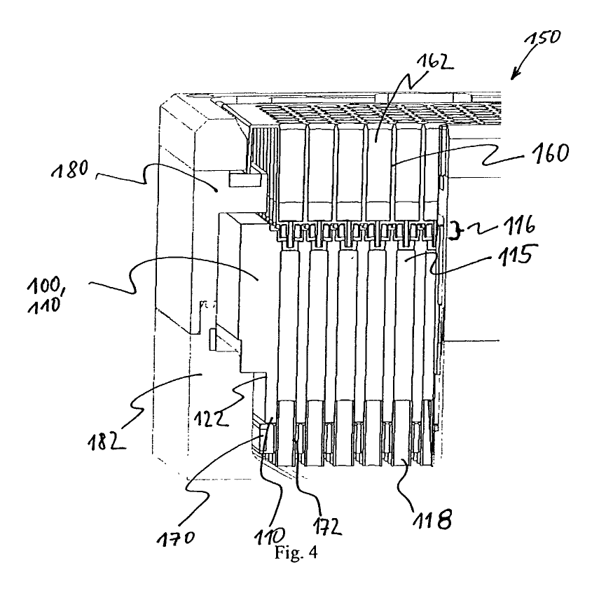

FIG. 4 is a cross-sectional view of a preparation apparatus 150 according to the invention in which an intermediate guide element 100 according to the invention is used. The preparation apparatus 150 comprises a multi-tube rack 160 (also known as a “QIAamp Plate”) which is secured to a vertically adjustable upper frame part 180 (also known as a “RoboVac Top”), and a hotplate 170 fixed to a lower frame part 182. Between the multi-tube rack 160 and the hotplate 170 is provided an intermediate guide element 100 according to the invention (also known as a “channelling block”) in such a way that each test tube 162 in the multi-tube rack 160 opens into a through-opening 115 of the intermediate guide element 100. The inlet regions 116 of the through-openings 115 are each enlarged in their diameter in a stepped configuration by means of a countersunk bore, similarly to the embodiment of the intermediate guide element according to the invention shown in FIG. 3. The outer contours of the outlet regions of the test tubes 162 in the multi-tube rack 160 are constructed accordingly, so that the test tubes 162 of the multi-tube rack 160 can be inserted to fit exactly in the through-openings 115.

Moreover, in the embodiment of the invention shown in FIG. 4, test tubes 118 are also provided on the bottom 112 of the main block 110 of the intermediate guide element 100, these test tubes 118 projecting from the main block 110 of the intermediate guide element 100 by roughly 1.2 times the thickness of the hotplate. In the assembled arrangement of the preparation apparatus 150 according to the invention as shown in FIG. 4, the tubes project through the hotplate 170 through bores 172 provided in the hotplate 170 and project from the underside of the hotplate by an amount corresponding to 0.2 times the thickness of the hotplate.

The side wall of the intermediate guide element 100 and the step 122 provided thereon serve to ensure that the intermediate guide element 100 can be gripped and positioned by automated gripping and positioning means.

The method of operation and function are as described in connection with FIG. 3.

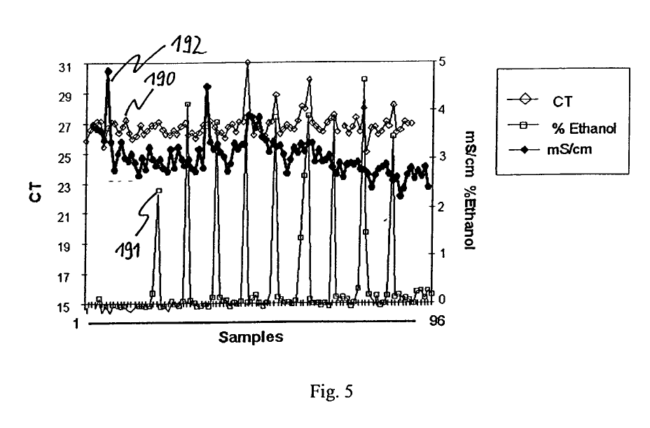

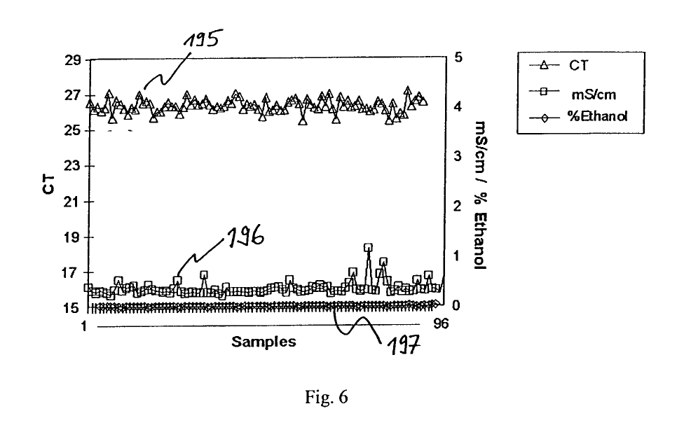

In FIGS. 5 and 6 preparation samples obtained with preparation apparatus known from the prior art were compared with samples obtained using a preparation apparatus according to the invention in terms of the parameters of amplifiability (TaqMan), ethanol concentration and salt content. These parameters are a measurement of the purity of the samples or, as a reciprocal value, a measurement of contamination or the formation of residues.

Both the samples prepared in FIG. 5 and those prepared in FIG. 6 were obtained by the QIAamp 96 virus procedure using SFV-RNA as template (10,000 c/prep).

FIG. 5 shows the results of samples obtained with a preparation apparatus known from the prior art. The 96 samples shown here (plotted on the x axis as samples) were prepared using the BioRobot 9604 with AirPore Tap for Dry Spin. The left-hand axis of the diagram shows the CT for the detection of SFV-RNA and the right-hand axis of the diagram shows the conductance of the eluates in mS/cm and the ethanol content in %. Trace 190 gives the CT value of the 96 samples, trace 192 gives the conductance of the eluates and trace 191 gives the ethanol content.

FIG. 6 similarly shows the results of samples obtained with a preparation apparatus according to the invention (prototype “BioRobot 8000 Clinical”) using an intermediate guide element according to the invention. Trace 195 gives the CT value of the 96 samples, trace 196 gives the conductance of the eluates and trace 197 gives the ethanol content.

The more stable CT's in FIG. 6 and the lower salt and ethanol concentration in the eluates of the samples of FIG. 6 and the substantially smaller range of fluctuations in the test results are clearly apparent; they can be put down to a substantially higher degree of purity and hence also reproducibility of the samples with one another. The preparation apparatus according to the invention thus demonstrably prevents the contamination of the samples and the formation of residues in the line system.

We claim:

1. An intermediate guide element (100) for use in a preparation apparatus (10, 150) comprising:
a main block (110) with a top (111) and a bottom (112) and at least one through-opening (115), preferably with a plurality of through-openings, preferably arranged parallel to one another, each through-opening (115) extending from the top (111) of the main block (110) to the bottom (112) of the main block (110) and
each through-opening (115) having an inlet region (116) and an outlet region (117),
the inlet region (116) being constructed so that the inlet region (116) can be attached to an end region of a feed line (22, 162), preferably the end region of the feed line can be inserted in the through-opening in an overlapping arrangement, and
attached to the outlet region (117) of each through-opening (115) is a singular guide element (118), the singular guide element (118) projecting beyond the main block (110) of the intermediate guide element (100) by a length which corresponds to at least one thickness of a hotplate (30, 170) arranged in the preparation apparatus (10, 150) on the underside (112) of the main block (110) of the intermediate guide element (100).

2. The intermediate guide element according to claim 1, wherein the through-openings (115) are constructed as bores.

3. The intermediate guide element according to claim 1, wherein the singular guide elements are tubes (118) which preferably fit into the outlet region of the associated through-opening.

4. The intermediate guide element according to claim 1, wherein the inlet region (116) is stepped, preferably so that it precisely fits the contour of the end region of the feed line.

5. The intermediate guide element according to claim 1, wherein the main block (110) has an encircling rim (120) on its top (111) and preferably also has at least one centring recess (121a, 121b).

6. A preparation apparatus (150) suitable for the fully automatic preparation of liquid samples, comprising
a feed element (160) comprising one or more feed lines (162), an intermediate guide element (100) according to one of the preceding claims, where the intermediate guide element has a number of through-openings (115) corresponding to the number of feed lines in the feed region and is adjacent to the feed element on the top of the main block, and
a hotplate (170) which is disposed on the bottom of the main block of the intermediate guide element (100) and which comprises a number of through-bores corresponding to the number of singular guide elements, the through-bores being arranged in the hotplate in such a way that in the assembled preparation apparatus the through-bores are aligned with the singular guide elements.

7. The preparation apparatus according to claim 6, wherein the intermediate guide element (100) is capable of being positioned fully automatically on the hotplate (170) of the preparation apparatus (150) by means of a gripping and positioning device.

8. The preparation apparatus according to one of claims 6 or 7, wherein the feed element is constructed as a single or multiple multi-tube rack (160) and preferably filters for filtering the liquid are disposed in the test tubes.

9. The preparation apparatus according to claim 8, wherein the feed element is fully automatically adjustable in height.

10. The preparation apparatus according to claim 6, wherein a vacuum can be applied to the side of the hotplate remote from the intermediate guide element.

11. An intermediate guide element (100) for use in a preparation apparatus (10, 150), comprising
a main block (110) with a top (111) and a bottom (112) and at least one through-opening (115), preferably with a plurality of through-openings, preferably arranged parallel to one another, the through-openings (115) extending from the top (111) of the main block (110) to the bottom (112) of the main block (110) and
the through-openings (115) each having an inlet region (116) and an outlet region (117),
the inlet region (116) being constructed so that the inlet region (116) can be attached to an end region of a feed line (22, 162), preferably the end region of the feed line can be inserted in the through-opening in an overlapping arrangement, and
the outlet regions (117) of the through-openings (115) each having a singular guide element (118).

12. The intermediate guide element (100) according to claim 11, wherein the ratio between the length of the through-openings (115) and the diameter of the through-openings (115) is in the range between 3:1 and 30:1, preferably in the range between 8:1 and 11:1.

13. The intermediate guide element (100) according to claim 11, wherein the singular guide element (118) projects beyond the main block (110) of the intermediate guide element (100) by a length which corresponds to at least one thickness of a hotplate (30, 170) arranged in the preparation apparatus (10, 150) on the bottom (112) of the main block (110) of the intermediate guide element (100).

14. The intermediate guide element according to one of claims 11 to 13, wherein the through-openings (115) are constructed as bores.

15. The intermediate guide element according to claim 14, wherein the singular guide elements are tubes (118) which preferably fit into the outlet region of the associated through-opening.

16. The intermediate guide element according to claim 14, wherein the inlet region (116) is stepped, preferably so that it precisely fits the contour of the end region of the feed line.

17. The intermediate guide element according to claim 14, wherein the main block (110) has an encircling rim (120) on its top (111) and preferably also has at least one centring recess (121a, 121b).

18. The intermediate guide element according to claim 14, wherein the intermediate guide element (100) is capable of being positioned fully automatically on a hotplate (170) of a preparation apparatus (150) by means of a gripping and positioning device.
